(19) United States
(12) Patent Application Publication
(10) Pub. No.: US 2011/0142891 A1
(43) Pub. Date: Jun. 16, 2011

(54) Cosmetic Pencil

(75) Inventors: Udo Pschirer, Selb (DE); Jürgen Raps, Hof (DE); Wolfgang Kaul, Heilsbronn (DE); Gerhard Lugert, Nürnberg (DE)

(73) Assignee: FABER CASTELL AG, Stein (DE)

(21) Appl. No.: 12/636,364

(22) Filed: Dec. 11, 2009

(51) Int. Cl. A61K 8/02; A61K 8/00; A61Q 1/02

(52) U.S. Cl. 424/401; 424/63

(57) ABSTRACT

The invention relates to a cosmetic pencil including a shaft with a first end and a second end, a reservoir disposed in the shaft, a liquid held in the reservoir, and a capillary application element in fluidic connection with the reservoir. The capillary application element is disposed at the first end of the shaft. A supply of a cosmetic preparation having solid, pasty, creamy or gel-like consistency is disposed at the second end of the shaft.

The invention relates to a cosmetic pencil with a shaft, a reservoir present therein and filled with a liquid and a capillary application element in connection with said reservoir and arranged at one end of the pencil. With such pencils it is possible to apply cosmetic liquids to the skin, for example to the lips, with high precision. Liquids suitable for capillary application systems must be relatively low-viscosity so that the capillary cavities of the application element do not become blocked. The choice and the fraction of the constituents in the liquid are thereby severely restricted. The applications that can be achieved therewith are often not optimal and still do not have the desired appearance. In order that the application produced with the help of the application element on the skin dries quickly, the liquid in most cases comprises a high fraction of readily volatile solvents, for example of alcohols. These have the tendency to withdraw sebum and moisture from the skin, meaning that it then feels dry and becomes taut.

Proceeding from this, it is the object of the invention to propose a cosmetic pencil which provides a remedy here.

This object is achieved by a cosmetic pencil according to claim 1, the use of such a pencil according to claim 9 and by a construction kit system according to claim 10 and more specifically by a supply of a cosmetic preparation having solid, pasty, creamy or gel-like consistency being arranged at one end of the shaft. The preparation can be applied in a second step to the skin, preferably to a part of the skin provided with the liquid. In this connection, all of those substances can be applied to the area of the skin which cannot be added to the liquid to be applied using the capillary application element, whether because they would make it too thick, contain particles that block the capillary channels of the application element or have to be applied subsequently for some other reason, which is the case, for example, in the case of a powder. On account of the solid, pasty, creamy or gel-like consistency of the preparation, this is subject to virtually no limitations as regards its ingredients. Furthermore, it is advantageous that no separate application element comprising for example a small sponge or a capillary active material is used, to which the composition to be applied in a second step would have to be adapted. Moreover, an additional application element would increase the production costs of the pencil.

A preparation present in the form of a solid lead ensures particularly simple handling. The application takes place here through direct contact of the lead with skin, without, as explained above, a further application element being required. Consequently, there is more space available for accommodating the preparation. Moreover, depending on the type of application to be produced, a lead can have different consistencies. If, for application of the preparation, an application element were envisaged, this and the consistency of the particular preparation would have to be matched to one another.

In a further preferred embodiment, a supply of the cosmetic preparation in the form of a paste, a cream or a gel is provided. For this, a, for example, bowl-shaped compartment in the receptacle is envisaged which is filled with the preparation in a type of cream canister. Removed using a finger, this can be applied to an area of the skin, preferably to one to which a, for example, coloring liquid has been applied beforehand with the help of the capillary application element.

Quite generally, the cosmetic liquid can comprise substances that care for and/or color the skin, primarily the lips and the eye area. The preparation for the second application comprises, for example, substances that generate a shine and/or care substances.

The invention is now illustrated in more detail by reference to the attached drawings. These show:

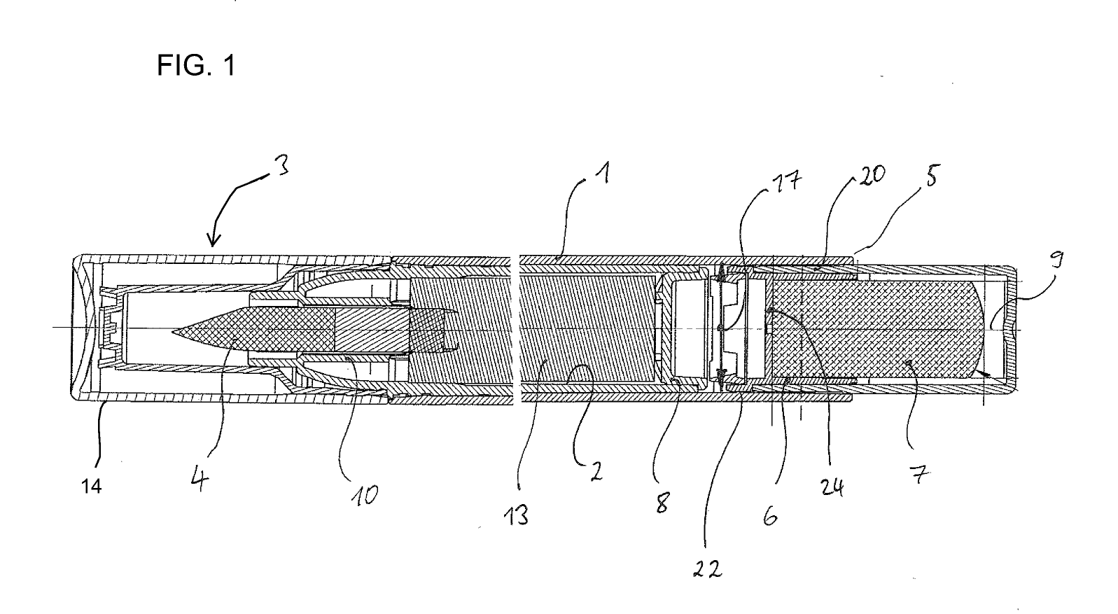

FIG. 1 a cosmetic pencil in longitudinal section view, at one end of which is arranged a capillary application element and at the other end of which is arranged a receptacle holding a solid lead,

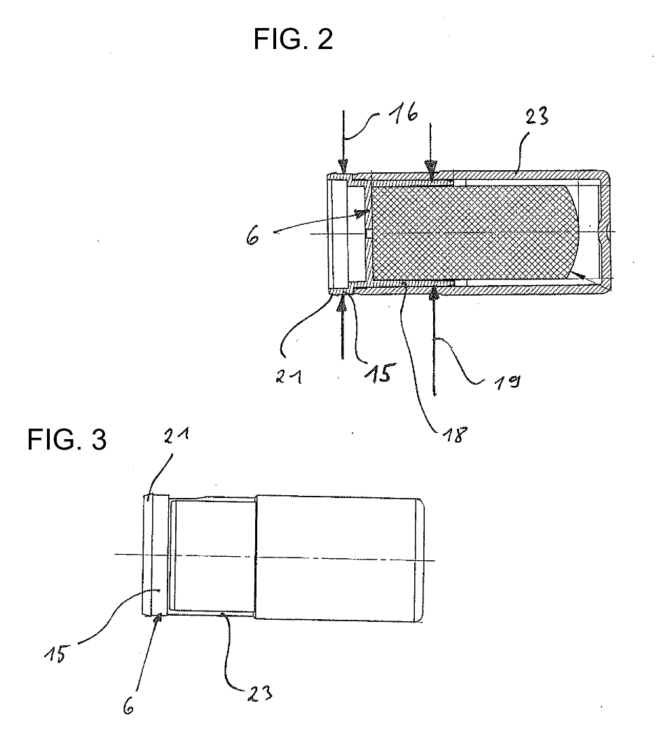

FIG. 2 the receptacle from FIG. 1 in longitudinal section view,

FIG. 3 the receptacle of FIG. 1 in side view,

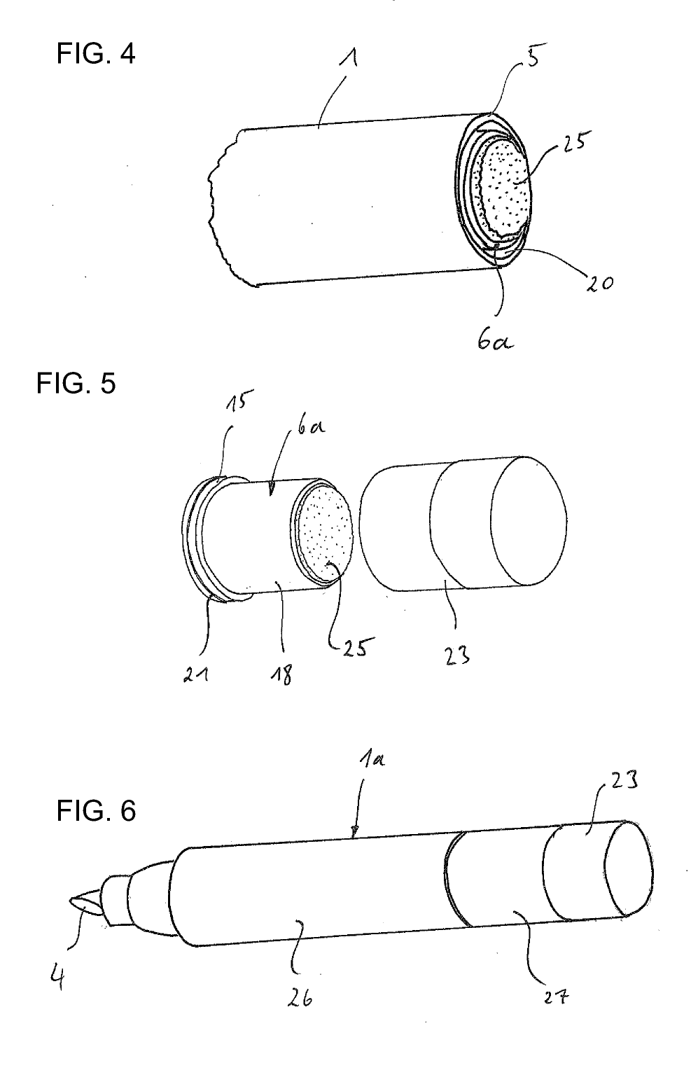

FIG. 4 the rear part of a cosmetic pencil in which a receptacle is arranged which comprises a pasty, creamy or gel-like preparation,

FIG. 5 the receptacle of FIG. 4 together with a closure cap,

FIG. 6 a cosmetic pencil, the shaft of which consists of two longitudinal sections joined together, where the one longitudinal section bears a capillary application element and the other longitudinal section bears a receptacle for a cosmetic preparation,

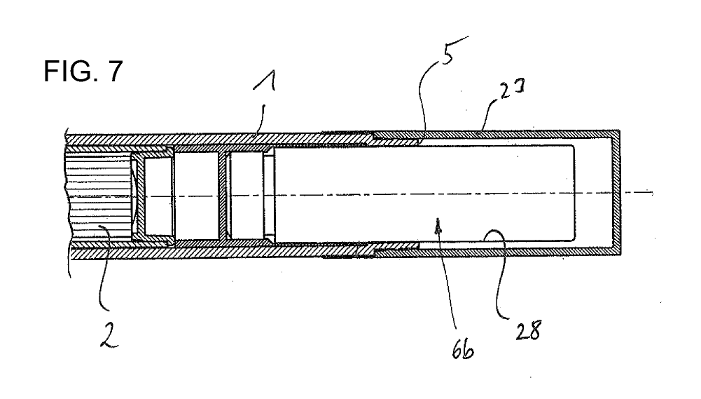

FIG. 7 the rear part of a cosmetic pencil with an alternatively designed receptacle.

A cosmetic pencil of the type according to the invention comprises a sleeve-like shaft 1, a reservoir 2 present therein for a cosmetic liquid, i.e. for a liquid that colors and/or cares for the skin, a capillary application element 4, which is in fluidic connection with the reservoir 2 and also a receptacle for a solid lead 7 arranged at the rear end 5 of the shaft 1. The receptacle is closed at its rear end by a base 8. At the front side, it leads into a tubular section 10 running coaxially to the central longitudinal axis 9 of the pencil, through which the approximately lead-shaped application element 4 travels. The application element consists, for example, of fibers extending in its longitudinal direction or of a sintered material. In the reservoir 2, preferably a capillary storage material 13 is present, which e.g. likewise consists of fibers or a sintered material. The application element 4 is protected against drying out and mechanical impairment by a closure cap 14 which can be slipped or screwed onto the front end of the stick.

In the shaft section adjoining the reservoir 2 to the back, the receptacle 6 is used in an axially fixed and preferably rotationally fixed position. Axial fixing can be brought about for example by a screw connection or—as in the case of FIGS. 1-3, by a snap connection. The snap connection or a comparable connection between receptacle 6 and shaft 1 does not necessarily have to be detachable. A detachable connection is expedient if the cosmetic pencil is part of a construction kit which, besides at least one cosmetic pencil, comprises a plurality of receptacles 6 supplied with different cosmetic preparations. The user can then put together a cosmetic pencil according to their own wishes, primarily choose an individual composition of liquid and preparation.

The receptacle in FIG. 1 is a substantially tubular-section-like part with two longitudinal sections of different diameter. A first longitudinal section 15, facing the front end 3 of the pencil, has an external diameter 16 which is only slightly smaller than the internal diameter 17 of the shaft 1. It bears a projection 21, which is held back with a projection 22 protruding from the inside surface of the shaft, in the sense of a snap connection. A second longitudinal section 18, adjoining the longitudinal section 15, of the receptacle 6 has an external diameter 19 which is dimensioned such that an annular gap 20 is formed between the longitudinal section 18 and the inside of the shaft 1. The front end of a closure cap 23 is inserted into this gap. The receptacle has a transverse wall 24. The solid lead 7 is inserted into the adjoining compartment of receptacle 6 in the direction of the rear pencil end 5, the length of the lead being dimensioned such that it protrudes from the receptacle 6 and the rear end 5 of the shaft.

FIGS. 4 and 5 show one embodiment of a cosmetic pencil in which the receptacle 6a has essentially the same design as the receptacle 6 in the embodiment described above. Except that no solid lead is inserted into the receptacle 6a, but its interior is filled with a pasty, creamy or gel-like substance 25 forming the cosmetic preparation. Also in the case of the embodiment in question, there is an annular gap 20 between the receptacle 6a and the pencil shaft 1, into which the closure cap 23 can be inserted. However, the closure cap 23 can quite generally be fixed to the pencil shaft 1 in another way, for example screwed onto a thread present on the outside of this or be simply slipped onto the outer periphery of the pencil (see FIG. 7).

In the case of the embodiment shown in FIG. 6, the reservoir 2 and the receptacle 6 (both not shown) are arranged in separate longitudinal sections 26, 27. The two shaft longitudinal sections 26, 27 are joined together, for example, by a screw connection or a snap connection. In the event that the cosmetic pencil is part of a construction kit, a detachable connection is envisaged which can be brought about e.g. in the specified manner.

Finally, FIG. 7 shows a cosmetic pencil of the type shown in FIG. 1, except that the receptacle 6b comprises a receiving sleeve 28 and a turning or sliding mechanism (not shown). Using this, the solid lead (not visible in FIG. 7), arranged when not being used entirely within the receiving sleeve 28, can be drawn out from said sleeve.

The cosmetic liquid contained in the reservoir serves, for example, to color the eyelids and the eyebrows. It comprises e.g. 0.1% by weight to 5% by weight of polyvinylpyrrolidone, 4% by weight to 25% by weight of an alcohol from the group consisting of ethanol, propanol, 2% by weight to 15% by weight of a humectant, 0.1% by weight to 5% by weight of polyoxyethylene glycerol fatty acid ester and/or polyether-modified polysiloxane and a colorant, where it has, in order to enable trouble-free application using the capillary application element 4, a viscosity of less than 50 mPas (Brookfield, 25° C.).

A further example of a liquid serving the same purpose comprises 3% by weight to 35% by weight of an aqueous plastics dispersion based on acrylate copolymer with a solids content of from 30% by weight to 50% by weight, 1% by weight to 20% by weight of an alcohol from the group consisting of ethanol, propanol, 2% by weight to 15% by weight of a humectant, 0.2% by weight to 5% by weight of polyoxyethylene glycerol fatty acid ester and/or polyether-modified polysiloxane and a colorant. The viscosity corresponds to that of the first-mentioned example. Very generally, care additives such as extracts from plants, for example aloe vera, collagen, pumpkin extract, ginseng, lecithin, panthenol, caffeine and hyaluronic acid are optionally present. The liquid can also exclusively serve for skincare, in which case it then does not comprise any colorants.

A lead mass suitable for the present purpose comprises, for example, 1-20% fatty acid salt, 5-40% of a lipophilic substance with a melting range below 50° C., 50-90% solids particles and up to 10% additives.

A further example which may be mentioned is a solid lead which comprises 25-65% of a saturated branched fatty alcohol, 5-35% wax and fatty acid derivative solid at 40° C., up to 12% adipic acid and/or citric acid ester, 30% 20% pigments and 2-15% additives such as thickeners, antioxidants, preservatives, emulsifiers etc.

In order to provide an application produced with the help of the capillary application element 4 with a powder layer, a lead with a powder-like consistency is envisaged. One such lead described for example in EP 1 291 399 B1 comprises a porous lead basic body, the cavities of which are filled with a solid or liquid phase. These and the aforementioned solid leads can likewise comprise colorants and/or care substances.

1-10. (canceled)

11. A cosmetic pencil comprising:
a shaft including a first end and a second end;
a reservoir disposed in said shaft;
a liquid held in said reservoir;
a capillary application element in fluidic connection with said reservoir, said capillary application element disposed at said first end of said shaft; and
a supply of a cosmetic preparation having solid, pasty, creamy or gel-like consistency disposed at said second end of said shaft.

12. The cosmetic pencil according to claim 11, further comprising a receptacle having a length at least partly extending within said shaft.

13. The cosmetic pencil according to claim 12, wherein said receptacle is detachably fixed in said shaft.

14. The cosmetic pencil according to claim 13, further comprising a closure cap covering said receptacle.

15. The cosmetic pencil according to claim 11, wherein:
said shaft includes two longitudinal sections;
said application element and said reservoir disposed in one of said two longitudinal sections; and
said preparation disposed in another one of said two longitudinal sections.

16. The cosmetic pencil according to claim 11, wherein said cosmetic preparation serves for covering said liquid after said liquid is applied to an area of skin using said capillary application element.

17. The cosmetic pencil according to claim 11, wherein said cosmetic preparation is formed as a solid lead.

18. The cosmetic pencil according to claim 17, further comprising a receptacle permitting said solid lead to be axially displaced.

19. A method of using a cosmetic pencil, which comprises:
obtaining a cosmetic pencil including:
a shaft including a first end and a second end,
a reservoir disposed in the shaft,
a liquid held in the reservoir,
a capillary application element in fluidic connection with the reservoir, the capillary application element disposed at the first end of the shaft, and
a supply of a cosmetic preparation having solid, pasty, creamy or gel-like consistency disposed at the second end of the shaft, and
first applying the liquid from the cosmetic pencil to an area of skin using the capillary application element and subsequently applying the cosmetic preparation from the cosmetic pencil to the area of the skin.

20. A construction kit system, comprising:
a plurality of receptacles holding different cosmetic preparations; and
a cosmetic pencil including:
a shaft including a first end and a second end;
a reservoir disposed in said shaft;
a liquid held in said reservoir;
a capillary application element in fluidic connection with said reservoir, said capillary application element disposed at said first end of said shaft; and
a supply of a cosmetic preparation having solid, pasty, creamy or gel-like consistency disposed at said second end of said shaft;
each of said plurality of receptacles being detachably fixable in said shaft of said cosmetic pencil.
